(19) United States
(12) Patent Application Publication
(10) Pub. No.: US 2005/0249721 A1
(43) Pub. Date: Nov. 10, 2005

(54) Compositions and methods for targeted biological delivery of molecular carriers

(76) Inventors: L. L. Houston, Bainbridge Island, WA (US); Stephen B. Hawley, San Diego, CA (US)

Correspondence Address: JONES DAY, 222 EAST 41ST ST, NEW YORK, NY, 10017, US

(21) Appl. No.: 10/510,170

(22) PCT Filed: Apr. 2, 2003
(86) PCT No.: PCT/US03/10233

§ 371 Date: Jun. 27, 2005

Related U.S. Application Data
(60) Provisional application No. 60/369,548, filed on Apr. 2, 2002.
(60) Provisional application No. 60/402,029, filed on Aug. 7, 2002.

(51) Int. Cl. A61K 48/00; A61K 39/395; A61K 38/17

(52) U.S. Cl. 424/130.1; 514/12; 514/44; 424/178.1

(57) ABSTRACT

The present invention relates to compositions and methods for the delivery of biologically-relevant moieties to subjects in need thereof. Particles comprising a physically entrapped targeting moiety, or a moiety that binds thereto, can promote paracellular transport, active transport, endocytosis or transcytosis into, through, out of and around, cells, tissues, and organs, optionally to an intracellular location. Such particles can provide targeted delivery of therapeutic, diagnostic, or imaging molecules.

### FIELD OF THE INVENTION

The invention is drawn to compositions and methods for the delivery of molecules into, through, out of and around, epithelial cells and layers and, optionally, to an intracellular location. The invention relates in part to particles adapted to provide targeted delivery of therapeutic, diagnostic, prophylactic, or imaging molecules by associating a targeting moiety that promotes paracellular transport, active transport, endocytosis and/or transcytosis with the particles.

### BACKGROUND OF THE INVENTION

The following description of the background of the invention is provided simply as an aid in understanding the invention and is not admitted to describe or constitute prior art to the invention.

A number of general methods have been described for delivering medically important molecules, including small molecules, nucleic acids, and/or protein or peptide compositions, to subjects in need thereof. Methods for improving bioavailability of such molecules include the use of prodrugs, encapsulation into liposomes or other particles, coadministration in uptake enhancing formulations, and targeting to specific tissues. For review see, e.g., Critical Reviews in Therapeutic Drug Carrier Systems, Stephen D. Bruck, ed., CRC Press, 1991. In particular, a number of particulates for the delivery of bioactive substances have been disclosed. Such particles are intended to enhance efficacy, e.g., by avoiding losses in activity caused by enzymatic degradation, protecting from pH extremes, providing a hydrophobic environment for poorly soluble molecules, and/or by enhancing uptake characteristics, etc. See, eg., U.S. Pat. Nos. 5,702,727; 5,620,708; 5,607,691; 4,610,896; 5,149,794; 6,197,349; 6,159,502; and 5,785,976. Such particles may act by (1) decreasing exposure to GI tract luminal proteases (following oral delivery), (2) decreasing exposure to complement defense components in the blood (during intravenous delivery), (3) minimizing dilution effects and/or inactivation of cargo proteins and/or nucleic acids due to binding to non-productive cell types, and (4) minimizing the amount of purified proteins and/or nucleic acids at sites distant from the site of interest, thereby minimizing potentially harmful toxic effects.

Molecules are trafficked into, out from and within a cell by various molecules. “Active transport” is a general term for the energy-dependent carriage of substances across a cell membrane. “Endocytosis” is a general term for the process of cellular internalization of molecules, i.e., processes in which cells take in molecules from their environment, either passively or actively. “Exocytosis” is a general term for processes in which molecules are passively or actively moved from the interior of a cell into the medium surrounding the cell. “Transcytosis” is a general term for processes in which molecules are transported from one surface of a cell to another. “Paracytosis” is a general term for processes in which molecules are transferred through the interstices between cells, often past tight junctions.

Active transport, endocytosis, exocytosis, transcytosis and paracytosis may involve or be mediated by receptors, molecules that are at least partially displayed on the surface of cells. Receptors have varying degrees of specificity; some are specific for a single molecule (e.g., a receptor specific for epidermal growth factor; or a receptor that specifically recognizes Ca⁺⁺); some are semi-specific (e.g., a receptor that mediates the cellular internalization of many members of a family of cellular growth factors, or a receptor that recognizes Ca⁺⁺, Mg⁺⁺ and Zn⁺⁺); or of limited specificity (e.g., a receptor that mediates the cellular internalization of any phosphorylated protein, or a receptor that recognizes any divalent cation). Other types of molecules that can cause or influence the entry of molecules into cells include, e.g., cellular pores, pumps, and coated pits. Pores such as gated channels and ionophores form a channel that extends through the cellular membrane and through which certain molecules can pass. Cellular pumps exchange one type of molecule within a cell for another type of molecule in the cell's environment. Coated pits are depressions in the cellular surface that are “coated” with bristlelike structures and which condense to surround external molecules; the condensed coated pits then “pinch off” to form membrane-bounded, coated vesicles within the cell.

A typical molecule that mediates forward transcytosis is the polymeric immunoglobulin receptor, or “pIgR,” which serves to convey protective antibodies (IgA and IgM immunoglobulins) from the circulatory system to the lumen of an organ. In forward transcytosis, pIgR molecules displayed on the basolateral side of the cell bind IgA molecules in the bloodstream, and pIgR:IgA complexes are then endocytosed, i.e., taken up into the cell and into a vesicle. The pIgR:IgA complexes are transported to the apical side of the cell, where they are displayed on the cell surface. Delivery of IgA into the lumen occurs when the pIgR portion of a pIgR:IgA complex is removed from the cell curface, e.g., by proteolysis. This event separates the pIgR molecule into two components: the “secretory component” (SC), which is released into the lumen, and which remains bound to IgA in order to protect IgA from degradation, and the “stalk,” which remains displayed, at least temporarily, on the apical surface of the cell. A third region, the “B region,” is removed with the secretory component by initial cleavage, but removed from secretory component by further proteolytic processing.

Surprisingly, ligands bound to molecules that mediate “forward” transcytosis (i.e. in the basolateral to apical direction) displayed on the apical side of a cell can undergo reverse transcytosis; that is, transcytosis in the opposite direction, (i.e., from the apical side of a cell to its basolateral side). In reverse transcytosis, pIgR molecules or portions thereof move from the apical surfaces of cells that line the lumen of an organ to the basolateral surfaces of these cells. pIgR-mediated reverse transcytosis may be used to deliver agents from a lumen (e.g., the interior of the gut or the airways of the lung) to the circulatory system or some other interior system, organ, tissue, portion or fluid of the body including by way of non-limiting example the lymphatic system, the vitreous humor, blood, cerebrospinal fluid, etc. A compound having an element that binds to a portion of pIgR that undergoes reverse transcytosis could, due to its association with the pIgR stalk, be carried to the basolateral side of a cell, where it would be contacted with and/or released into the bloodstream.

The polyimmunoglobulin receptor (pIgR) is reviewed by Mostov and Kaetzel, Chapter 12 in: Mucosal Immunology, Academic Press, 1999, pages 181-211 (1999). U.S. Pat. No. 6,020,161 to Wu et al. is drawn to pIgR polypeptides and polynucleotides that encode pIgR polypeptides. U.S. Pat. No. 5,484,707 to Goldblum et al. is drawn to methods for monitoring organ rejection in an animal based on the concentration of the free secretory component of (SC) pIgR, Published PCT patent applications WO 98/30592 and WO 99/20310, both to Hein et al., and U.S. Pat. No. 6,045,774 to Hiatt et al., are drawn to synthetic proteins that mimic IgA molecules and are thus associated with the proteolytically generated secretory component (SC) of pIgR, U.S. Pat. No. 6,072,041 to Davis et al. is drawn to fusion proteins that are directed to the secretory component of pIgR. Ferkol et al., Am. J. Respir. Crit. Care Med. 161:944-951, 2000, is stated to describe a fusion protein consisting of a sFv directed to the secretory component (SC) of human pIgR and an human alpha-(1)-antitrypsin. U.S. Pat. No. 6,042,833 to Mostov et al. is drawn to a method by which a ligand that binds to a portion of a pIgR molecule is thereby internalized into, or transported across, a cell expressing or displaying pIgR. U.S. Pat. No. 6,083,741, to Hart et al., entitled “Internalization of DNA, Using Conjugates of Poly-lysine and an Intergrin Receptor Ligand,” combines this technique with the use of an integrin receptor ligand. Zhang et al. (Cell 102:827-837, 2000) states that pIgR translocates bacteria (specifically, Streptococcus pneumoniae) across nasopharyngeal epithelial cells in the apical to basolateral (reverse) direction.

U.S. patent applications related to the present application include U.S. Provisional Patent Application No. 60/199,423 entitled “Compositions Comprising Carriers and Transportable Complexes,” filed Apr. 23, 2000; PCT/US01/09699, entitled “Ligands Directed to the Non-Secretory Component, Non-Stalk Region of pIgR and Methods of Use Thereof,” filed Mar. 27, 2000; and PCT/US01/30832 entitled “Compositions and Methods for Identifying, Characterizing, Optimizing and Using Ligands to Transcytotic Molecules,” filed Oct. 10, 2001.

Each publication and patent application in the foregoing Background section is hereby incorporated by reference in its entirety, including all tables, figures, and claims.

### SUMMARY OF THE INVENTION

The present invention relates generally to compositions for targeted biological delivery of medically relevant molecules, and methods for their use. The invention relates in part to particles or capsules adapted to provide delivery of therapeutic, diagnostic, prophylactic, or imaging molecules (referred to herein as “medically-relevant moieties”) into, through, out of and around, epithelial cells and layers and, optionally, to an intracellular location. Such particles and capsules comprise a targeting moiety that specifically binds to a cell surface component that promotes paracellular transport, active transport, endocytosis or transcytosis, and a biologically active moiety. In particular, a portion of the targeting moiety, or an anchor moiety that binds to the targeting moiety, is associated with a particle or capsule having physical dimensions compatible with cellular uptake. While any method of associating the targeting or anchor moiety with the particle or capsule is contemplated by the present invention, it is preferred that all or a portion of the targeting or anchor moiety is physically entrapped within the particle or capsule. Upon binding to the cell surface component, the particle or capsule, containing a payload of one or more therapeutic, diagnostic, or imaging molecules, is transported to a location within a cell, tissue, or organ where the payload is released from within the particle or capsule.

The particles of the present invention provide for improved bioavailability of the payload molecules, most preferably therapeutic moieties selected from the group consisting of a polypeptide, an antibody, an antibody fragment, a single-chain variable region fragment, a small molecule, an oligonucleotide, an oligosaccharide, a polysaccharide, a cyclic polypeptide, a peptidomimetic, and an aptamer. The particles and capsules of the present invention can be used to deliver such payload molecules via common routes of delivery, such as an oral, nasopharyngeal, oropharyngeal, pulmonary, mucosal, vaginal, transcutaneous, or rectal route.

Thus, in a first embodiment, the present invention relates to methods for preparing a therapeutic composition. These methods comprise associating (i) a portion of a targeting moiety, or an anchor moiety that binds to the targeting moiety, and (ii) a medically-relevant moiety, with a particle or capsule having physical dimensions compatible with cellular uptake. The targeting moiety specifically binds to a cell surface component that promotes paracellular transport, active transport, endocytosis or transcytosis. Each particle or capsule may comprise one or more distinct targeting moieties that specifically bind to different cell surface component (or different binding regions on the same cell surface component); and/or one or more different medically-relevant moieties.

In various preferred embodiments, all or a portion of the targeting or anchor moiety is physically entrapped within the particle or capsule; the medically-relevant moiety is entrapped within the particle or capsule; the cell surface component is present on epithelial cells, most preferably enterocytes; the cell surface component is present on endothelial cells; the targeting moiety and the therapeutic moiety are not bound to one another; the targeting moiety and the therapeutic moiety are covalently or noncovalently bound to one another, the targeting moiety is selected from the group consisting of an antibody, an antibody fragment, a single-chain variable region fragment, a small molecule, oligonucleotide, oligosaccharide, polysaccharide, cyclic polypeptide, peptidomimetic, and aptamer; the cell surface component is selected from the group consisting of pIgR, pIgR stalk, pIgR B region, transferrin receptor, vitamin B12 receptor, FcRn, members of the PGDF and VEGF receptor families (e.g., Flt-1, Flk-1, Flt-4), and low density lipoprotein receptor; and/or the medically-relevant moiety is selected from the group consisting of a polypeptide, an antibody, an antibody fragment, a single-chain variable region fragment, a small molecule, an oligonucleotide, an oligosaccharide, a polysaccharide, a cyclic polypeptide, a peptidomimetic, and an aptamer.

In those particles and capsules in which the targeting moiety (or moieties) are attached via an anchor moiety, the anchor moiety may comprise a first region entrapped within the particle and a second region protruding from the surface of the particle for binding to the targeting moiety (or moieties). In various preferred embodiments, the first region is selected from the group consisting of a polypeptide, a nucleic acid, a poly(ethylene oxide), a peptidomimetic, a cyclic peptide, a oligosaccharide, a polysaccharide, an aptamer, and a dextran; and the second region is selected from the group consisting of a polypeptide, an antibody, an antibody fragment, a single-chain variable region fragment, a small molecule, an oligonucleotide, an oligosaccharide, a polysaccharide, a cyclic polypeptide, a peptidomimetic, and an aptamer. In particularly preferred embodiments the second region is a polypeptide sequence that forms a coiled-coil with a complementary polypeptide sequence on said targeting moiety. Alternatively, in those particles in which the targeting moiety (or moieties) are not attached via an anchor moiety, the targeting moiety may comprise a first region entrapped within the particle and a second region protruding from the surface of the particle that specifically binds to said cell surface component.

Pores may be produced by swelling the particle or capsule, and the first region of the anchor moiety or targeting moiety, and/or the medically-relevant moiety, may be entrapped in the pores by reducing the swelling (eg., by altering the buffer in which the particle is suspended). During such a physical entrapment step, the particle or capsule may comprise pores having physical dimensions capable of accepting said first region, but incapable of accepting said second region, whereby the anchor and/or targeting moiety is entrapped in an oriented fashion. Alternatively, the physical nature of the internal portions of the particle or capsule may be selected to entrap such moiety (or moieties) with a preferred orientation (e.g., a hydrophobic particle interior, or a particular charge or charge density may be used to preferentially orient the first region during entrapment and/or to entrap the medically-relevant moiety). In still another alternative, the moiety (or moieties) may be entrapped without any particular orientation being preferred. In such a physical entrapment step, sufficient anchor and/or targeting moieties are entrapped such that a sufficient number of moieties exhibit the proper orientation.

The first region of the anchor moiety or targeting moiety, and/or the medically-relevant moiety, may also be entrapped in the particle during polymerization of material forming the particle. As discussed in the proceeding paragraph, during such an entrapment the physical nature of the internal portions of the particle may be selected to entrap such moiety (or moieties) with a preferred orientation, or the moiety (or moieties) may be entrapped without any particular orientation being preferred using sufficient anchor and/or targeting moieties such that a sufficient number of moieties exhibit the proper orientation.

The term “therapeutic composition” as used herein refers to a composition for treating a disease, or one or more symptoms thereof, in a human or non-human animal subject. Preferably, such a composition comprises one or more therapeutic moieties reversibly entrapped in a particle, as described below.

The term “diagnostic composition” as used herein refers to a composition for identifying the presence or absence of one or more markers related to the presence or absence of a disease. Preferably, a diagnostic composition comprises an antibody.

The term “prophylactic composition” as used herein refers to a composition for preventing a disease, or one or more symptoms thereof, in a human or non-human animal subject. Preferably, such a composition comprises one or more therapeutic moieties reversibly entrapped in a particle, as described below. Most preferably, a prophylactic composition is an immunogenic composition that acts as a vaccine to prevent a disease or symptom of a disease.

The term “imaging composition” refers to a composition for enhancing contrast in an X-ray, MR, CT, nuclear, and/or acoustic (e.g., ultrasound) procedure. Typical particulate imaging compositions are disclosed in, e.g., U.S. Pat. Nos. 6,251,366; 6,203,777; 5,976,500; 5,928,626; and 5,670,135, each of which is hereby incorporated by reference in its entirety, including all tables, figures, and claims

The term “therapeutic moiety” as used herein refers to a molecule or portion of a molecule that, when introduced into a living organism, modifies one or more functions of the organism. Preferred therapeutic moieties are small molecules, prodrugs, polypeptides, antibodies, antibody fragments, single-chain variable region fragments, polynucleotides, oligonucleotides, oligonucleotide analogs, oligosaccharides, polysaccharides, cyclic polypeptides, peptidomimetics, and aptamers.

As used herein, the term “small molecule” refers to compounds having molecular mass of less than 3000 Daltons, preferably less than 2000 or 1500, still more preferably less than 1000, and most preferably less than 600 Daltons. Preferably but not necessarily, a small molecule is not an oligopeptide.

As used herein, the term “polypeptide” refers to a covalent assembly comprising at least two monomeric amino acid units linked to adjacent amino acid units by amide bonds. An “oligopeptide” is a polypeptide comprising a short amino acid sequence (i.e., 2 to 10 amino acids). An oligopeptide is generally prepared by chemical synthesis or by fragmenting a larger polypeptide. Examples of polypeptide drugs include, but are not limited to, therapeutic antibodies, insulin, parathyroid hormone, polypeptide vaccines, and antibiotics such as vancomycin. Novel polypeptide drugs may be identified by, e.g., phage display methods.

As used herein, the term “antibody” refers to an immunoglobulin molecule obtained by in vitro or in vivo generation of an immunogenic response, and includes both polyclonal, monospecific and monoclonal antibodies, and antigen binding fragments thereof (e.g., Fab fragments). An “immunogenic response” is one that results in the production of antibodies directed to one or more proteins after the appropriate cells have been contacted with such proteins, or polypeptide derivatives thereof, in a manner such that one or more portions of the protein function as epitopes.

As used herein, the term “single-chain variable region fragment” or “sFv” refers to a variable, antigen-binding determinative region of a single antibody light chain and antibody heavy chain linked together by a covalent linkage having a length sufficient to allow the light and heavy chain portions to form an antigen binding site. Such a linker may be as short as a covalent bond; preferred linkers are from 2 to 50 amino acids, and more preferably from 5 to 25 amino acids.

As used herein, the term “polynucleotide” refers to molecule comprising a covalent assembly of nucleotides linked typically by phosphodiester bonds through the 3′ and 5′ hydroxyls of adjacent ribose units. An “oligonucleotide” is a polynucleotide comprising a short base sequence (i.e., 2 to 10 nucleotides). Polynucleotides include both RNA and DNA, may assume three-dimensional shapes such as hammerheads, dumbbells, etc., and may be single or double stranded. Polynucleotide drugs can include ribozymes, ribozymes, and polynucleotide vaccines,

As used herein, the term “oligonucleotide analog” refers to a molecule that mimics the structure and function of an oligonucleotide, but which is not a covalent assembly of nucleotides linked by phosphodiester bonds. Peptide nucleic acids, comprising purine and pyrimidine bases linked via a backbone linkage of N-(2-aminoethyl)-glycine units, is an example of an oligonucleotide analog.

The term “polysaccharide” as used herein refers to a carbohydrate comprising 2 or more covalently-linked saccharide units. An “oligosaccharide” is a polysaccharide comprising a short saccharide sequence (i.e., 2 to 10 saccharide units).

As used herein, the term “cyclic polypeptide” refers to a molecule comprising a covalent assembly of monomeric amino acid units, each of which is linked to at least two adjacent amino acid units by amide bonds to form a macrocycle.

As used herein, the term “peptidomimetic” refers to a molecule that mimics the structure and function of an polypeptide, but which is not a covalent assembly of amino acids linked by amide bonds. A peptoid, which is a polymer of N-substituted glycine units, is an example of a peptidomimetic.

The term “aptamer” as used herein refers to polynucleotides that bind to non-polynucleotide target molecules (e.g., a polypeptide or small molecule).

The term “targeting moiety” as used herein refers to any molecular structure that is directed to (specifically binds) a molecule to which it is targeted. The term “specifically binds” is not intended to indicate that the targeting moiety binds exclusively to its intended target. Rather, a targeting moiety specifically binds if its affinity for its intended target is about 2-fold greater when compared to its affinity for a non-target molecule. Preferably the affinity of the targeting moiety will be at least about five fold, preferably 10 fold, more preferably 25-fold, even more preferably 50-fold, and most preferably 100-fold or more, greater for a target molecule than its affinity for a non-target molecule. A composition comprising such a targeting moiety would be referred to as being “adapted to specifically bind” to the target molecule. Preferred targeting moieties can be selected from the group consisting of a polypeptide, a recombinant polypeptide, an antibody, an antibody fragment, a single-chain variable region fragment, a small molecule, an oligonucleotide, an oligosaccharide, a polysaccharide, a cyclic polypeptide, a peptidomimetic, and an aptamer, as these terms are defined herein.

Most preferably, a targeting moiety is directed to a protein selected from the group consisting of a cell surface receptor, most preferably a pIgR molecule; and a cell surface molecule other than a receptor, most preferably a pIgR stalk molecule, a pIgR B region, and/or a pIgR secretory component molecule. Such elements confer the property of being able to undergo apical or basolateral endocytosis, apical or basolateral exocytosis, and/or forward or reverse transcytosis in cells displaying a pIgR molecule, a pIgR stalk molecule, a pIgR B region, and/or a pIgR secretory component molecule. By way of non-limiting example, the pIgR may be a simian pIgR, a murine pIgR, a rodent pIgR, a rabbit pIgR, a bovine pIgR, or a human pIgR.

The term “cell surface component” as used herein refers to a molecule, or a portion of a molecule, present on an external surface of a cell and that is accessible to targeting moieties placed in contact with the cell. A targeting moiety is said to be “cell-specific” if it is directed to a cell surface component that is exclusively or preferentially 30 displayed on the surface of a cell of a particular cell type or tissue. Preferred cell surface components include, but are not limited to, receptors such as pIgR, transferrin receptor, vitamin B12 receptor, FcRn, intergrins low density lipoprotein receptor; cargo carrier ragments such as pIgR stalk, members of the PGDP, FGF, and VEGF receptor families e.g., Flt-1, FIk-1, Flt-4, FGFR1, FGFR2, FGFR3, FGFR4), and surface antigens.

A cell surface component is said to “promote paraccllular transport, active transport, endocytosis, or transcytosis” if a composition comprising a targeting moiety that specifically binds to the cell surface component is transported into, around, or through a cell (depending on the type of transport involved) at a higher rate compared to a similar composition lacking the targeting moiety.

One or more molecules, e.g., targeting moieties, anchor moieties, and/or medically-relevant moieties are associated with the particles or capsules of the present invention in order to provide various targeting, therapeutic, diagnostic, etc., properties to the composition. Numerous methods for associating such molecules with a particle or capsule are known to those of skill in the art, including covalent attachment to a component of the particle or capsule, electrostatic attachment to a component of the particle or capsule (e.g., adsorption to a surface), physical entrapment of all or a portion of the molecule by the particle or capsule, and/or indirect binding to a component of the particle or capsule (e.g., via an avidin-biotin or similar binding pair, where the particle or capsule is attached to one member of the binding pair, and a molecule to be associated is attached to the other member of the binding pair. These examples are not meant to be limiting.

In preferred embodiments, various moieties are associated with particles and capsules such that a portion of the moiety (e.g., the portion of a targeting moiety that specifically binds to a cell surface component) is properly exposed on the surface of the particle or capsule. Proper exposure may be provided, for example, by binding to the particle or capsule through a portion of the targeting moiety that is sufficiently separated from the portion that specifically binds to the cell surface component such that steric hindrance of the binding interaction is avoided. Alternatively, a linker can be used to provide a linkage between the targeting moiety and the particle or capsule surface. Preferably, the targeting moiety is attached to a linker of between about 5 Å and about 1000 Å, more preferably between about 10 Å and about 500 Å, even more preferably between about 50 Å and about 300 Å, and most preferably between about 75 Å and about 200 Å. The term “about” in this context refers to +/−10% of a given measurement.

The term “physically entrapped” as used herein refers to a molecule or a portion of a molecule that is embedded within a particle of the invention, and which is not required for formation of the particle itself. A physically entrapped molecule is not retained within or on the particle by covalent, electrostatic, or hydrophobic interactions, but rather by exhibiting a size that is too great to exit pores within the particle or that follows a pathway (e.g., a labyrinthine pathway) within the particle that prevents its release. Such a physically entrapped molecule is not released by the particle so long as the particle retains structural integrity, and may be released from the particle only if the particle is partially or completely degraded or otherwise changes in physical dimension (e.g., by swelling).

The term “particle” as used herein refers to an integral structural element having dimensions of between about 1000 μm and about 1 nm in overall dimension capable of retaining one or more molecules for delivery to a subject (“a payload”). Such particles are preferably porous and/or biodegradable, and most preferably selectively porous and/or biodegradable (ie., only in certain environments). Preferred particles have physical dimensions compatible with cellular uptake, e.g., about 10 μm to about 10 nm, most preferably about 1 μm to about 5 nm. The term “microparticle” refers to particles from 1 to 1000 μm, while “nanoparticle” refers to particles less than 1 μm in size. The term “about” in this context refers to +/−10% of a given dimension. Such particles include polymeric microparticles, virions, liposomes, lipoprotein particles, lipid emulsions, and lipid suspensions, and may comprise an internal polymer matrix, an internal fluid, or an amorphous or crystalline internal phase. See, e.g., U.S. Pat. No. 6,197,349. Particles may be formulated for topical, ingestible, injectable, and inhaled applications.

The term “capsule” as used herein refers to a subset of particles that are vesicular structural elements having dimensions of between about 1000 μm and about 1 nm in overall dimension capable of retaining one or more molecules for delivery to a subject (“a payload”). One or more molecules for delivery are confined to a central cavity surrounded by an outer shell, such as a polymer or lipid membrane. Preferred capsules have physical dimensions compatible with cellular uptake, e.g., about 10 μm to about 10 nm, most preferably about 1 μm to about 5 nm. The term “microcapsule” refers to capsules from 1 to 1000 μm, while “nanocapsule” refers to capsules less than 1 μm in size. The term “about” in this context refers to +/−10% of a given dimension. Capsules can include “particle-in-particle” and “particle-in-coating” structures. See, e.g. Soppimath et al., J. Controlled Release 70: 1-20 (2001); McPhail et al., Intl. J. Pharmaceutics 200: 73 86 (2000).

The term “anchor moiety” as used herein refers to any molecular structure that may be physically entrapped in a particle or capsule and that is or may be covalently or noncovalently bound to a targeting moiety. For example, an anchor moiety may comprise a first region that is a polypeptide, a nucleic acid, a poly(ethylene oxide), a peptidomimetic, a cyclic peptide, a oligosaccharide, a polysaccharide, an aptamer, or a dextran, that is entrapped within a particle, and a second region that projects from the particle and that binds to a complementary region on a targeting moiety. Any binding interaction may be employed by the skilled artisan for binding between the anchor moiety and the targeting moiety. In preferred embodiments, the second region comprises a nucleic acid sequence that is complementary to a sequence in the targeting moiety; the second region comprises an aptamer that binds to a region present on the targeting moiety; the second region comprises a region that binds to an aptamer present on the targeting moiety; the second region comprises an amino acid sequence that forms a coiled-coil domain with an amino acid sequence in the targeting moiety, and the second region comprises one or more cysteine residues that form one or more disulfide bonds with cysteine residues in the targeting moiety. These examples are not meant to be limiting.

In another aspect, the present invention relates to the therapeutic, diagnostic, or imaging compositions described herein. These compositions preferably comprise particles or capsules comprising one or more targeting moiety (or moieties) that specifically bind to a cell surface component that promotes paracellular transport, active transport, endocytosis and/or transeytosis, and one or more biologically-relevant moiety (or moieties). As described above, the targeting moiety (or moieties), or anchor moiety (or moieties) that bind to the targeting moiety, and the biologically-relevant moiety (or moieties) are preferably physically entrapped within the particle or capsule. Most preferably, the particle or capsule is of a size compatible with cellular uptake, e.g., about 10 μm to about 10 nm, most preferably about 1 μm to about 5 nm.

The compositions of the present invention may be used to deliver biologically-relevant moieties to any cell capable of paracellular transport, active transport, endocytosis, and/or transcytosis. Examples of cells that may be targeted by such compositions include epithelial cells (e.g., squamous, transitional cuboidal, and columnar epithelial cells), most preferably epithelial cells lining the gastrointestinal tract (e.g., enterocytes), the alveolae, the trachea, the nasopharynx, the bronchial tree, the oropharynx, the vaginal tract, the skin or mucosal surfaces, and/or the rectum; endothelial cells; and endothelial cells, most preferably those cells lining the circulatory and/or the lymphatic systems. Thus, in another aspect, the present invention relates to methods of delivering a therapeutic, diagnostic, or imaging compositions described herein to a subject in need thereof.

The term “subject” as used herein refers to a human or a non-human animal. Thus, the methods and compositions described herein can be used for both medical and veterinary purposes.

The biological environment presented to such compositions when delivered to a subject can be hostile to biologically-relevant moieties present within the composition. In the case of polypeptides and oligonucleotides for example, proteases and nucleases are often present in biological systems (e.g., blood, the gastrointestinal tract, etc.). Additionally, the gastrointestinal tract can expose unprotected biologically-relevant moieties to extremes of pH (about pH 1-pH 4.5) It is therefore preferred that the compositions of the present invention protectively retain the biologically-relevant moieties prior to cellular uptake.

The term “protectively retain” is not meant to indicate that all of the biologically-relevant moieties originally present in a particle or capsule be bioavailable; instead, the term indicates that at least a percentage of the biologically-relevant moieties be bioavailable in a functional form and in an amount sufficient to have its intended biological effect. In preferred embodiments, the compositions of the present invention provide at least about 1% bioavailability, more preferably about 5% bioavailability, still more preferably at least about 10% bioavailability, even more preferably at least about 25% bioavailability, still more preferably at least about 50% biavailability, and most preferably at least about 75% bioavailability. The term “about” in this context refers to +/−10% of a given percentage (e.g., about 10% means from 9% to 11%).

In preferred embodiments, the particle or capsule is made of a biodegradable material, such that the cargo of the nanoparticle may be released into a cell or into the blood or tissues, thus rendering the biologically-relevant moieties originally present in a particle or capsule bioavailable.

The term “bioavailability” refers to the extent to which an biologically-relevant moiety (or the active metabolite of a pro-form of such a moiety, e.g., a prodrug) enters the general circulation, and is thereby available to its site of action. A bioavailability of 5% means that 5% of the moiety delivered to the subject (e.g., orally) enters the circulation in functional form.

The summary of the invention described above is not limiting and other features and advantages of the invention will be apparent from the following detailed description of the preferred embodiments, as well as from the claims.

### DETAILED DESCRIPTION OF THE INVENTION

The present invention describes compositions for targeted biological delivery of medically relevant molecules, and methods for their production and use. In particular, particles and/or capsules adapted to provide delivery of therapeutic, diagnostic, or imaging molecules (referred to herein as “medically-relevant moieties”) into, through, out of and around, epithelial cells and layers and, optionally, to an intracellular location are described.

As described in detail herein, such particles and capsules comprise one or more targeting moieties that specifically bind to a cell surface component that promotes paracellular transport, active transport, endocytosis and/or transcytosis, together with a biologically active moiety. In preferred embodiments, all or a portion of the targeting moiety, or an anchor moiety that binds to the targeting moiety, is physically entrapped within the particle. Upon binding to the cell surface component, the particle, containing a payload of one or more therapeutic, diagnostic, or imaging molecules, is transported to a location within a cell, tissue, or organ where the payload is released from within the particle.

Preparation of Particles and Capsules

Methods for producing particulate administration systems for delivery of biologically-relevant molecules are well known to those of skill in the art. Such particles are preferably porous and/or biodegradable so that molecules (e.g., drugs, vaccines, vitamins, polypeptides, antibodies, etc.) contained within the particle may be released once delivered into the inside of a cell or through the cell into the circulation; however, nonporous and/or nonbiodegradable particles (e.g., liposomes) are also known to those of skill in the art. Preferred particles and capsules, including microparticles, nanoparticles, microcapsules, and nanocapsules are disclosed in, e.g., U.S. Pat. Nos. 5,702,727; 5,620,708; 5,607,691; 4,610,896; 5,149,794; 6,197,349; 6,159,502; 5,785,976; Chiu et al., Biomaterials 23: 1103-12 (2002); Andrianov et al., Biomaterials 19: 109-115 (1998); Soppimath et al., J. Controlled Release 70: 1-20 (2001); McPhail et al., Intl. J. Pharmaceutics 200: 73-86 (2000); Müller et al., Eur. J. Pharmaceut. Biopharmaceut. 50: 161-177 (2000); Franssen et al., J. Controlled Release 60: 211-21 (1999); Prokop et al., Biotechnol. and Bioeng. 75: 228-232 (2001); Allémann et al., Adv. Drug Deliv. Rev. 34: 171-89 (1998); Vinogradov et al., Adv. Drug Deliv. Rev. 54: 135-47 (2002); Jung et al., Eur. J. Pharmaccut. Biopharmaceut. 50: 147-60 (2000); Martin et al., Biomaterials 19: 69-76 (1998); Vervoort et al., Intl. J. Pharmaceutics 172: 137-45 (1998); J. Controlled Release 65: 49-54 (2000); Davda and Labhasetwar, Intl. J. Pharmaceutics 223: 51-9 (2002); Düzgünes and Nir, Adv. Drug Deliv. Rev. 40: 3-18 (1999); Nagayasu et al., Adv. Drug Deliv. Rev. 40: 75-87 (1999); Leroueil-Le Verger et al., Eur. J. Pharmaceut. Biopharmaceut. 46: 137-143 (1998); Breton et al., Biomaterials 19: 271-81 (1998); Konan et al., Intl. J. Pharmaceutics 233: 239-52 (2002); Duncan et al, Eur. Polymer J. 37: 1821-6 (2001); and Stenekes et al., Biomaterials 22: 1891-8 (2001), each of which is hereby incorporated by reference in its entirety. Such delivery systems may be formulated for topical, ingestible, injectable, and inhaled applications.

As discussed above, one or more molecules, e.g., targeting moieties, anchor moieties, and/or medically-relevant moieties may be associated with particulate administration systems by numerous methods for associating such molecules with a particle or capsule are known to those of skill in the art, including covalent attachment to a component of the particle or capsule, electrostatic attachment to a component of the particle or capsule, physical entrapment of all or a portion of the molecule by the particle or capsule, and/or indirect binding to a component of the particle or capsule.

For example, Duncan et al, Eur. Polymer J. 37: 1821-6 (2001), discloses mixing of a drug (heparin) with a monomeric structural unit (PHEMA), followed by immobilization of the drug during glutaraldehyde crosslinking to form a hydrogel. Allémann et al., Adv. Drug Deliv. Rev. 34: 171-89 (1998), discloses incorporation of a polypeptide into a polymeric particle by swelling/drying the particle, followed by encapsulation in a bioadhesive. Leroueil-Le Verger et al., Eur. J. Pharmaceut. Biopharmaceut. 46: 137-143 (1998), discloses preparing a colloidal suspension of a drug and a polymeric nanoparticle in acetone, followed by evaporation of the acetone to entrap the particle. Vinogradov et al., Adv. Drug Deliv. Rev. 54: 135-47 (2002) discloses binding of oligonucleotide molecules within nanogels, followed by coupling of a targeting molecule via an avidin-biotin binding pair.

Substances to be physically entrapped within particulate administration systems can be entrapped within the particle by two general mechanisms. First, the substances may be dispersed (e.g., dissolved, suspended, solubilized) into the mixture of materials (e.g., polymerizable monomers, stabilizers, lipids, etc.) used to form the particles. In these methods, the substances of interest become entrapped during particle formation. In performing such methods, the orientation of substances entrapped in the particle may be controlled, for example by incorporating an aliphatic or steroidal tail onto an otherwise hydrophilic molecule and employing a particle comprising a hydrophobic region. In this case, the entrapped substances will orient much like amphipathic lipids in a lipid bilayer. For example, Jung et al., Eur. J. Pharmaceut. Biopharmaceut 50: 147-60 (2000) discloses method for providing polymeric nanoparticles having a “core-corona” structure having distinct hydrophilic and hydrophobic domains. Alternatively, substances may be entrapped without any particular preferred orientation. In such a case, one can expect a certain percentage of the substances to be randomly entrapped in the proper orientation.

In a second alternative, substances to be entrapped within particulate administration systems can be entrapped within the particle after the particle has been made. For example, hydrogel or other particles may be swelled by altering the environment of the particle (e.g., temperature, solvent conditions, etc.). See, e.g., U.S. Pat. No. 6,303,148. Entrapment may be effected by mixing the substances to be entrapped with the swelled particles, following by shrinkage to complete the entrapment. Again, substances may be entrapped using methods providing a preferred orientation, or substances may be randomly entrapped.

In those particles in which the targeting moiety (or moieties) is (are) attached via an anchor moiety, the anchor moiety may be entirely contained within the particle. In such embodiments, a targeting moiety can be covalently or noncovalently linked to a biologically-relevant moiety that is itself entrapped within a particle (e.g., an antibody directed to a cell surface molecule such as pIgR can be linked to a therapeutic antibody). Thus, the biologically-relevant moiety can act as an anchor moiety. Alternatively, the anchor moiety may comprise a first region entrapped within the particle and a second region protruding from the surface of the particle for binding to the targeting moiety.

In either case, the anchor moiety and the targeting moiety (or moieties) may be bound to one another using binding interactions that are well known to those of skill in the art. Appropriate binding interactions include, but are not limited to, antibody-antigen, receptor-hormone, avidin-biotin pairs, streptavidin-biotin, metal-chelate, small molecule/polynucleotide (see, e.g., Dervan, Bioorg. Med. Chem 9: 2215-35 (2001); Zahn and Dervan, Bioorg. Med. Chem. 8: 2467-74 (2000); polynucleotide/complementary polynucleotide (e.g., dimeric and trimeric helices), aptamer/small molecule, aptamer/polypeptide, coiled-coil, and polynucleotide/polypeptide (e.g. zinc finger, helix-tum-helix and helix-loop-helix motifs that bind to DNA sequences). As an example of a binding interaction that may be formed between an anchor moiety and a targeting moiety, the following is a discussion of a protein-protein interaction known to those of skill in the art as a “leucine zipper.”

Leucine zippers are polypeptide motifs typically seen in DNA binding proteins. Such motifs are approximately 35 amino acids in length, and comprise a leucine every seven residues. Two leucine zipper motifs form a coiled-coil in which the leucine residues on the two strands interdigitate, forming hydrophobic interactions with one another to stabilize the dimeric structure. For use in the present invention, a leucine zipper may be attached to a targeting moiety (e.g., an antibody directed to a cell surface molecule such as pIgR) by chemical crosslinking or by standard molecular biology techniques; and be a second region of an anchor moiety with, e.g., a (poly)ethylene oxide polyethylene glycol) first region that becomes entrapped within a particle.

In an alternative to the use of an anchor moiety, a portion of the targeting moiety itself can be used to entrap the targeting moiety within the particle. In these embodiments, a portion other than the binding portion of the targeting moiety can be entrapped, either during or after assembly of the particle, in the same manners as discussed above. The skilled artisan will understand that a targeting moiety can be covalently linked to a biologically-relevant moiety. In these embodiments, the biologically-relevant moiety can be viewed in a sense as that portion of the targeting moiety entrapped within the particle.

Targeting of Particles for Transport

The compositions of the present invention may be used to deliver biologically-relevant moieties to any cell capable of paracellular transport, active transport, endocytosis, and/or transcytosis. Numerous ligands are known to enter or exit biological systems by binding to a component that mediates transport of the ligand to or from the cell surface. Examples of such ligands include diphtheria toxin, pseudomonas toxin, cholera toxin, ricin, concanavalin A, certain viruses (Rous sarcoma virus, adenovirus, etc.), transferrin, low density lipoprotein, transcobalamin (vitamin B12), insulin, epidermal growth factor, growth hormone, thyroid stimulating factor, calcitonin, glucagon, prolactin, lutenizing hormone, thyroid hormone, platelet derived growth factor, VEGFs, IgA, and IgM. This list is not meant to be limiting. Particles comprising one or more targeting moieties can utilize the same transport mechanisms used by natural ligands to improve bioavailability of molecules. See, e.g., PCT/US01/30832 entitled “Compositions and Methods for Identifying, Characterizing, Optimizing and Using Ligands to Transcytotic Molecules;” filed Oct. 10, 2001. A discussion of immunglobulin transport mediated by pIgR follows as an example of mediated transport useful in the present invention.

A pIgR molecule has several structurally and functionally distinct regions that are defined as follows. It has been mentioned above that, in the art, a pIgR molecule is generally described as consisting of two different, loosely defined regions called the “stalk” and the “secretory component” (SC). A pIgR molecule binds polymeric immunoglobulins (IgA or IgM) on the basolateral side, and then transports the immunoglobulin to the apical side. Proteolytic cleavage of pIgR takes place on the apical side of an epithelial cell between the SC and the stalk, the former of which remains bound to and protects the immunoglobulins, and the latter of which remains bound to the apical membrane (see “Mucosal Immunoglobulins” by Mestecky et al. in: Mucosoal Immunology, edited by P. L. Ogra, M. E. Lamm, J. Bienenstock, and J. R. McGhee, Academic Press, 1999). Ligands bound to “stalks” displayed on the apical side of a cell can undergo reverse transcytosis, i.e., transcytosis in the opposite direction of forward transcytosis, i.e., from the apical side of a cell to its basolateral side. In reverse transcytosis, pIgR molecules or portions thereof move from the apical surfaces of cells that line the lumen of an organ to the basolateral surfaces of these cells. See, e.g., U.S. Pat. No. 6,072,041, which is hereby incorporated by reference in its entirety, including all tables, figures, and claims.

Extracellular domains 1 through 6 of pIgR molecules from several species are indicated in FIG. 3 of Piskurich et al. (J. Immunol. 154:1735-1747, 1995). In rabbit pIgR, domains 2 and 3 are encoded by a single exon that is sometimes deleted by alternative splicing. A transmembrane domain is also present in pIgR, as is an intracellular domain. The intracellular domain contains signals for transcytosis and endocytosis. Domains of a pIgR molecule that are of particular interest in the present disclosure include but are not limited to domain 5, domain 6, the transmembrane domain and the intracellular domain.

As used herein, the term “stalk” refers to a molecule having an amino acid sequence derived from a pIgR, but which does not comprise amino acid sequences derived from the secretory component. A stalk molecule comprises amino acid sequences that remain bound to the apical membrane following the apical proteolytic cleavage when such cleavage occurs and amino acid sequences required for such cleavage. Preferred stalk molecules confer one or more transcytotic properties to a ligand bound thereto. Most preferred are stalk molecules that confer the ability to undergo apical to basolateral transcytosis to a ligand bound thereto.

As used herein, the term “B region” refers to a non-secretory component, non-stalk region of pIgR. After transport to the apical surface of an epithelial cell, pIgR undergoes an initial cleavage, releasing a portion of pIgR into the extracellular space, with residual stalk region remaining accessable on the cell surface. The released portion undergoes further degradation by proteolytic enzymes to generate secretory component. The region that is degraded following release is referred to as the B region of pIgR. See, e.g., WO 01/72846, which is hereby incorporated by reference in its entirety. Ligands that bind to this B region can exhibit one or more transcytotic properties as described herein. B region can be an advantageous choice for directing transcytosis of a composition as described herein, as intact pIgR on a cell surface will bind to the composition without competition from secretory component.

Several preferred embodiments are described herein in which a molecule used to configure a particle for delivery comprises a first region “coupled” in some sense to a second region. For example, an anchor moiety may comprise a first region within a particle and a second region external to the particle; a targeting moiety may comprise a first region within a particle and a second region external to the particle; a targeting moiety may comprise a first region that binds to anchor moiety, e.g., by forming a binding interaction such as a coiled-coil; and/or a targeting moiety may be covalently bound to a medically relevant moiety. The skilled artisan will understand that such regions may be simply two portions of a single molecule (an example of two such regions may be an Fc region and an Fab region on an antibody), or two molecules linked by a tethering “linker moiety.” Numerous methods are available to the skilled artisan to provide such “coupled” molecules.

For example, any two components (e.g., two components independently selected from the group consisting of a polypeptide, an antibody, an antibody fragment, a single-chain variable region fragment, a small molecule, an oligonucleotide, an oligosaccharide, a polysaccharide, a cyclic polypeptide, a peptidomimetic, and an aptamer, a poly(ethylene oxide), a dextran, etc.). Thus, a targeting moiety and a nucleic acid sequence that is complementary to a sequence in the anchor moiety, or an aptamer that binds to a region present on the anchor moiety, or an amino acid sequence that forms a coiled-coil domain with an amino acid sequence in the anchor moiety, etc., may be chemically cross-linked by a linker having chemistry compatible with a site on each component. Crosslinkers are well known to those of skill in the art, and may be obtained commercially (see, e.g., Pierce Chemical Company Catalog and Handbook 1994-95, pages O-90 through O-110, which is hereby incorporated by reference) or synthesized as needed.

Alternatively, in cases where both components are peptides, the components may be coupled “genetically”; that is, the first and second regions may be expressed as a fusion protein. For example, U.S. Pat. No. 6,072,041 to Davis et al. is drawn to fusion proteins that are directed to the secretory component of pIgR Ferkol et al., Am. J. Respir. Crit. Care Med. 161:944-951, 2000, discloses a fusion protein consisting of a sFv directed to the secretory component (SC) of human pIgR and an human alpha (1)—antitrypsin. U.S. Pat. No. 6,042,833 to Mostov et al. discloses “genetic fusions” and “fusion proteins” that include ricin A, poly-(L)-Lys, or a phage surface protein. In this manner, for example, a coiled-coil sequence may be attached to an anchor moiety and a targeting moiety to provide the complementarity necessary to achieve binding between the two moieties.

Compositions

The compositions of the present invention provide for delivery of biologically-relevant moieties, ie., therapeutic, diagnostic, prophylactic, or imaging molecules to a subject in need thereof. The compositions of the invention can further comprise other chemical components, such as diluents and excipients. A “diluent” is a chemical compound diluted in a solvent, preferably an aqueous solvent, that facilitates dissolution of the therapeutic agent in the solvent, and it may also serve to stabilize the biologically active form of the chimeric pIgR-targeting protein or one or more of its components. Salts dissolved in buffered solutions are utilized as diluents in the art. For example, preferred diluents are buffered solutions containing one or more different salts. A preferred buffered solution is phosphate buffered saline (particularly in conjunction with compositions intended for pharmaceutical administration), as it mimics the salt conditions of human blood. Since buffer salts can control the pH of a solution at low concentrations, a buffered diluent rarely modifies the biological activity of a biologically active peptide.

An “excipient” is any more or less inert substance that can be added to a composition in order to confer a suitable property, for example, a suitable consistency or to form a drug. Suitable excipients and carriers include, in particular, fillers such as sugars, including lactose, sucrose, mannitol, or sorbitol cellulose preparations such as, for example, maize starch, wheat starch, rice starch, agar, pectin, xanthan gum, guar gum, locust bean gum, hyaluronic acid, casein potato starch, gelatin, gum tragacanth, polyacrylate, methyl cellulose, hydroxypropylmethyl-ellulose, sodium carboxymethylcellulose, and/or polyvinylpyrrolidone (PVP). If desired, disintegrating agents can also be included, such as cross-linked polyvinyl pyrrolidone, agar, or alginic acid or a salt thereof such as sodium alginate. Other suitable excipients and carriers include hydrogels, gellable hydrocolloids, and chitosan. Chitosan microspheres and microcapsules can be used as carriers. See WO 98/52547 (which describes microsphere formulations for targeting compounds to the stomach, the formulations comprising an inner core (optionally including a gelled hydrocolloid) containing one or more active ingredients, a membrane comprised of a water insoluble polymer (e.g., ethylcellulose) to control the release rate of the active ingredient(s), and ari outer layer comprised of a bioadhesive cationic polymer, for example, a cationic polysaccharide, a cationic protein, and/or a synthetic cationic polymer; U.S. Pat. No. 4,895,724. Typically, chitosan is cross-linked using a suitable agent, for example, glutaraldehyde, glyoxal, epichlorohydrin, and succinaldehyde. Compositions employing chitosan as a carrier can be formulated into a variety of dosage forms, including pills, tablets, microparticles, and microspheres, including those providing for controlled release of the active ingredient(s). Other suitable bioadhesive cationic polymers include acidic gelatin, polygalactosamine, polyamino acids such as polylysine, polyhistidine, polyornithine, polyquaternary compounds, prolamine, polyimine, diethylaminoethyldextran (DEAE), DEAE-imine, DEAE-methacrylate, DEAE-acrylamide, DEAE-dextran, DEAE-cellulose, poly-p-aminostyrene, polyoxethane, copolymethacrylates, polyamidoamines, cationic starches, polyvinylpyridine, and polythiodiethylaminomethylethylene.

The compositions of the invention can be formulated in any suitable manner. Suitable formulations include dry and liquid formulations. Dry formulations include freeze dried and lyophilized powders, which are particularly well suited for aerosol delivery to the sinuses or lung, or for long term storage followed by reconstitution in a suitable diluent prior to administration. Other preferred dry formulations include those wherein a composition according to the invention is compressed into tablet or pill form suitable for oral administration or compounded into a sustained release formulation. As those in the art will appreciate, the compositions of the invention can be placed into any suitable dosage form. Pills and tablets represent some of such dosage forms. The compositions can also be encapsulated into any suitable capsule or other coating material, for example, by compression, dipping, pan coating, spray drying, etc. Suitable capsules include those made from gelatin and starch. In turn, such capsules can be coated with one or more additional materials, for example, and enteric coating, if desired. Liquid formulations include aqueous formulations, gels, and emulsions.

Some preferred embodiments concern compositions that comprise a bioadhesive, preferably a mucoadhesive, coating. A “bioadhesive coating” is a coating that allows a substance (e.g., a according to the invention) to adhere to a biological surface or substance better than occurs absent the coating. A “mucoadhesive coating” is a preferred bioadhesive coating that allows a substance, for example, a composition according to the invention, to adhere better to mucosa occurs absent the coating. For example, micronized particles (e,g., particles having a mean diameter of about 5, 10, 25, 50, or 100 μm) can be coated with a mucoadhesive. The coated particles can then be assembled into a dosage form suitable for delivery to an organism. Preferably, and depending upon the location where the cell surface transport moiety to be targeted is expressed, the dosage form is then coated with another coating to protect the formulation until it reaches the desired location, where the mucoadhesive enables the formulation to be retained while the chimeric pIgR-targeting proteins interact with the target cell surface transport moiety.

The particular amount of biologically active component to be delivered will depend on many factors, including the effect to be achieved, the type of organism to which the composition is delivered, delivery route, dosage regimen, and the age, health, and sex of the organism. As such, the particular dosage is left to the ordinarily skilled artisan's discretion.

Depending on the mode of delivery employed, the context-dependent functional entity can be delivered in a variety of pharmaceutically acceptable forms. For example, the context-dependent functional entity can be delivered in the form of a solid, solution, emulsion, dispersion, micelle, liposome, and the like, incorporated into a pill, capsule, tablet, suppository, areosol, droplet, or spray. Pills, tablets, suppositories, areosols, powders, droplets, and sprays may have complex, multilayer structures and have a large range of sizes. Aerosols, powders, droplets, and sprays may range from small (1 micron) to large (200 micron) in size.

The compositions of the present invention can comprise the usual non-toxic, pharmaceutically acceptable carriers for tablets, pellets, capsules, suppositories, solutions, emulsions, suspensions, and any other form suitable for use. The carriers which can be used include glucose, lactose, mannose, gum acacia, gelatin, mannitol, starch paste, magnesium trisilicate, talc, corn starch, keratin, colloidal silica, potato starch, urea, medium chain length triglycerides, dextrans, and other carriers suitable for use in manufacturing preparations, in solid, semisolid, or liquid form. In addition auxiliary, stabilizing, thickening and coloring agents and perfumes may be used. Examples of a stabilizing dry agent includes triulose, preferably at concentrations of 0.1% or greater (See, e.g., U.S. Pat. No. 5,314,695).

Pharmaceutical formulations of particular interest in the context of the invention include, but are not limited to, those taught is U.S. Pat. No. 5,254,342, entitled “Compositions and methods for enhanced transepithelial and transendothelial transport o[f] active agents” to Shen et al.; and U.S. Pat No. 6,110,456, “Oral delivery o[f] adeno-associated viral vectors.”

The compositions of the present invention may be used in therapeutic, prophylactic, diagnostic, and/or imaging methods.

Nucleic acids for use as biologically-relevant moieties in the present invention include, but are not limited to, catalytic nucleic acids, e.g., ribozymes; structural nucleic acids, e.g., ribosomal RNA (rRNA); transfer RNA (tRNA); antisense nucleic acids, e.g., antisense oligonucleotides; aptamers; nucleic acids decoys, e.g., dsDNA comprising sequences to which DNA-binding proteins,specifically bind; and expression elements that direct the in vivo production of a biologically active nucleic acid or polypeptide. The biologically active nucleic acids of the invention may be of any shape, form or topology including, but not limited to, double-stranded (ds), including A-, B- and Z-DNA; single-stranded (ss); mixed ds and ss; linear, circular; hybrid (e.g., RNA:DNA hybrids); supercoiled; compacted; nicked; complexed with other nucleic acids and/or polypeptides; etc.

Polypeptides for use as biologically-relevant moieties in the present invention include include, but are not limited to, (1) endogenous polypeptides that are missing, deficient, mutated or underexpressed in a patient suffering from a particular disease or disorder, (2) endogenous polypeptides that can be overexpressed to achieve a biological effect; and (3) exogenous proteins such as recombinant peptides. Some therapies involving polypeptides of type (1) are designed so as to be therapeutic for inborn errors of metabolism and include, by way of non-limiting example, enzyme replacement (e.g., Factor IX in the case of hemophilia B, and phenylalanine hydroxylase in the case of phenylketonuria) therapy, and protein or factor replacement (e.g., Factor VIII in the case of hemophilia A, and insulin in the case of Type I diabetes) therapy. See, e.g., Dai et al., Proc. Natl. Acad. Sci. USA 89:10892-10895, 1992; and Wang et al., Proc. Natl. Acad. Sci. USA 96:3906-3910, 1999.

An additional exemplary list of suitable compounds for use as biologically-relevant moieties in the present invention is provided below.

analgesics/antipyretics (e.g., aspirin, acetaminophen, ibuprofen, naproxen sodium, buprenorphine hydrochloride, propoxyphene hydrochloride, propoxyphene napsylate, meperidine hydrochloride, hydromorphone hydrochloride, morphine sulfate, oxycodone hydrochloride, codeine phosphate, dihydrocodeine bitartrate, pentazocine hydrochloride, hydrocodone bitartrate, levorphanol tartrate, diflunisal, trolamine salicylate, nalbuphine hydrochloride, mefenamic acid, butorphanol tartrate, choline salicylate, butalbital, phenyltoloxamine citrate, diphenhydramine citrate, methotrimeprazine, cinnamedrine hydrochloride, meprobamate, and the like);

antimigraine agents (e.g., ergotamine tartrate, propanolol hydrochloride, isometheptene mucate, dichloralphenazone, and the like);

sedatives/hypnotics (e.g., barbiturates (e.g., pentobarbital, pentobarbital sodium, secobarbital sodium), benzodiazapines (e.g., flurazepam hydrochloride, triazolam, tomazeparm, midazolam hydrochloride, and the like);

antianginal agents (e.g., beta-adrenergic blockers, calcium channel blockers (e.g., nifedipine, diltiazem hydrochloride, and the like), nitrates (e.g., nitroglycerin, isosorbide dinitrate, pentaerythritol tetranitrate, erythrityl tetranitrate, and the like));

antianxiety agents (e.g., lorazepam, buspirone hydrochloride, prazepam, chlordiazepoxide hydrochloride, oxazepam, clorazepate dipotassium, diazepam, hydroxyzine pamoate, hydroxyzine hydrochloride, alprazolam, droperidol, halazepam, chlormezanone, and the like);

antipsychotic agents (e.g., haloperidol, loxapine succinate, loxapine hydrochloride, thioridazine, thioridazine hydrochloride, thiothixene, fluphenazine hydrochloride, fluphenazine decanoate, fluphenazine enanthate, trifluoperazine hydrochloride, chlorpromazine hydrochloride, perphenazine, lithium citrate, prochlorperazine, and the like);

antimanic agents (e.g., lithium carbonate),

antiarrhythmics (e.g., bretylium tosylate, esmolol hydrochloride, verapamil hydrochloride, amiodarone, encainide hydrochloride, digoxin, digitoxin, mexiletine hydrochloride, disopyramide phosphate, procainamide hydrochloride, quinidine sulfate, quinidine gluconate, quinidine polygalacturonate, flecainide acetate, tocainide hydrochloride, lidocaine hydrochloride, and the like);

antiarthritic agents (e.g., phenylbutazone, sulindac, penicillamine, salsalate, piroxicam, azathioprine, indomethacin, meclofenamate sodium, gold sodium thiomalate, ketoprofen, auranofin, aurothioglucose, tolmetin sodium, and the like);

antigout agents (e.g., colchicine, allopurinol, and the like);

anticoagulants (e.g., heparin (a repeating disaccharide unit of D-glucosamine and uronic acid linked by 1→4 interglycosidic bond having a molecular weight of between about 6000 to about 40000 daltons, usually between 12000 and 15000 daltons), heparin sodium, warfarin sodium, and the like);

thrombolytic agents (e.g., urokinase, streptokinase, altoplase, and the like);

antifibrinolytic agents (e.g., aminocaproic acid);

hemorheologic agents (e.g., pentoxifylline);

antiplatelet agents (e.g., aspirin, empirin, ascriptin, and the like);

anticonvulsants (e.g., valproic acid, divalproate sodium, phenytoin, phenytoin sodium, clonazepam, primidone, phenobarbitol, phenobarbitol sodium, carbamazepine, amobarbital sodium, methsuximide, metharbital, mephobarbital, mephenytoin, phensuximide, paramethadione, ethotoin, phenacemide, secobarbitol sodium, clorazepate dipotassium, trimethadione, and the like);

antiparkinson agents (e.g., ethosuximide, and the like);

antidepressants (e.g., doxepin hydrochloride, amoxapine, trazodone hydrochloride, amitriptyline hydrochloride, maprotiline hydrochloride, phenelzine sulfate, desipramine hydrochloride, nortriptyline hydrochloride, tranylcypromine sulfate, fluoxetine hydrochloride, doxepin hydrochloride, imipramine hydrochloride, imipramine pamoate, nortriptyline, amitriptyline hydrochloride, isocarboxazid, desipramine hydrochloride, trimipramine maleate, protriptyline hydrochloride, and the like);

antihistamines/antipruritics (e.g., hydroxyzine hydrochloride, diphenhydramine hydrochloride, chlorpheniramine maleate, brompheniramine maleate, cyproheptadine hydrochloride, terfenadine, clemastine fumarate, triprolidine hydrochloride, carbinoxamine maleate, diphenylpyraline hydrochloride, phenindamine tartrate, azatadine maleate, tripelennamine hydrochloride, dexchlorpheniramine maleate, methdilazine hydrochloride, trimprazine tartrate and the like);

antihypertensive agents (e.g., trimethaphan camsylate, phenoxybenzamine hydrochloride, pargyline hydrochloride, deserpidine, diazoxide, guanethidine monosulfate, minoxidil, rescinnamine, sodium nitroprusside, rauwolfia serpentina, alseroxylon, phentolamine mesylate, reserpine, and the like);

agents useful for calcium regulation (e.g., calcitonin, parathyroid hormone, and the like);

antibacterial agents (e.g., amikacin sulfate, aztreonam, chloramphenicol, chloramphenicol palmitate, chloramphenicol sodium succinate, ciprofloxacin hydrochloride, clindamycin hydrochloride, clindamycin palmitate, clindamycin phosphate, metronidazole, metronidazole hydrochloride, gentamicin sulfate, lincomycin hydrochloride, tobramycin sulfate, vancomycin hydrochloride, polymyxin B sulfate, colistimethate sodium, colistin sulfate, and the like);

antifungal agents (e.g., griseofulvin, keloconazole, and the like);

antiviral agents (e.g., interferon gamma, zidovudine, amantadine hydrochloride, ribavirin, acyclovir, and the like);

antimicrobials (e.g., cephalosporins (e.g., cefazolin sodium, cephradine, cefaclor, cephapirin sodium, ceftizoxine sodium, cefoperazone sodium, cefotetan disodium, cefutoxime azotil, cefotaxime sodium, cefadroxil monohydrate, ceftazidime, cephalexin, cephalothin sodium, cephalexin hydrochloride monohydrate, cefamandole nafate, cefoxitin sodium, cefonicid sodium, ceforanide, ceftriaxone sodium, ceftazidime, cefadroxil, cephradine, cefuroxime sodium, and the like), penicillins (e.g., ampicillin, amoxicillin, penicillin G benzathine, cyclacillin, ampicillin sodium, penicillin G potassium, penicillin V potassium, piperacillin sodium, oxacillin sodium, bacampicillin hydrochloride, cloxacillin sodium, ticarcillin disodium, azlocillin sodium, carbenicillin indanyl sodium, penicillin G potassium, penicillin G procaine, methicillin sodium, nafcillin sodium, and the like), erythromycins (e.g., erythromycin ethylsuccinate, erythromycin, erythromycin estolate, erythromycin lactobionate, erythromycin siearate, erythromycin ethylsuccinate, and the like), tetracyclines (e.g., tetracycline hydrochloride, doxycycline hyclate, minocycline hydrochloride, and the like), and the like);

anti-infectives (e.g., GM-CSF);

bronchodialators (e.g., sympathomimetics (e.g., epinephrine hydrochloride, metaproterenol sulfate, terbutaline sulfate, isoetharine, isoetharine mesylate, isoetharine hydrochloride, albuterol sulfate, albuterol, bitolterol, mesylate isoproterenol hydrochloride, terbutaline sulfate, epinephrine bitartrate, metaproterenol sulfate, epinephrine, epinephrine bitartrate), anticholinergic agents (e.g., ipratropium bromide), xanthines (e.g., aminophylline, dyphylline, metaproterenol sulfate, aminophylline), mast cell stabilizers (e.g., cromolyn sodium), inhalant corticosteroids (e.g., flurisolidebeclomethasone dipropionate, beclomethasone dipropionate monohydrate), salbutamol, beclomethasone dipropionate (BDP), ipratropium bromide, budesonide, ketotifen, salmeterol, xinafoate, terbutaline sulfate, triamcinolone, theophylline, nedocromil sodium, metaproterenol sulfate, albuterol, flunisolide, and the like);

cytokines (e.g., interleukins IL-1, IL-2, IL-3, IL-4, IL-5, IL-6, IL-7, IL-8, IL-9, IL-10, IL-11 , IL-12, IL-13, IL-14, IL-15, IL-16, IL-17, IL-18, IL-19, IL-20, interferons alpha, beta, and gamma,

growth factors (e.g., growth hormone, insulin-like growth factor 1 and 2 (IGF-1 and IGF-2), vascular endothelial growth factor (VEGF), granulocyte colony stimulating factor (GCSF), glucose sensitive factor (GSF), platelet derived growth factor (PGDF), epidermal growth factor (EGF), fibroblast growth factor (FGF), transforming growth factor beta (TGF-beta));

tumor necrosis factor (TNF), TNF receptor, TNF inhibitors (e.g., etanercept and infliximab), and antibodies to TNF or its receptor;

hormones (e.g., androgens (e.g., danazol, testosterone cypionate, fluoxymesterone, ethyltostosterone, testosterone enanihate, methyltestosterone, fluoxymesterone, testosterone cypionate), estrogens (e.g., estradiol, estropipate, conjugated estrogens), progestins (e.g., methoxyprogesterone acetate, norethindrone acetate), corticosteroids (e.g., triamcinolone, betamethasone, betamethasone sodium phosphate, dexamethasone, dexamethasone sodium phosphate, dexamethasone acetate, prednisone, methylprednisolone acetate suspension, triamcinolone acetonide, methylprednisolone, prednisolone sodium phosphate methylprednisolone sodium succinate, hydrocortisone sodium succinate, methylprednisolone sodium succinate, triamcinolone hexacatonide, hydrocortisone, hydrocortisone cypionate, prednisolone, fluorocortisone acetate, paramethasone acetate, prednisolone tebulate, prednisolone acetate, prednisolone sodium phosphate, hydrocortisone sodium succinate, and the like), thyroid hormones (e.g., levothyroxine sodium) and the like), erythropoietin (EPO), and the like;

hypoglycemic agents (e.g., purified or recombinant human insulin, purified or recombinant beef insulin, purified or recombinant pork insulin, glyburide, chlorpropamide, glipizide, tolbutamide, tolazamide, and the like);

hypolipidemic agents (e.g., clofibrate, dextrothyroxine sodium, probucol, lovastatin, niacin, and the like);

proteins (e.g., DNase, alginase, superoxide dismutase, lipase, anmtibodies, and the like, synthetic proteins, recombinant proteins, chimeric proteins (i.e., comprising domains derived from more than one protein));

nucleic acids (e.g., sense or anti-sense nucleic acids encoding any protein suitable for delivery by inhalation, including the proteins described herein, and the like);

agents useful for erythropoiesis stimulation (e.g., erythropoietin);

antiulcer/antireflux agents (e.g., famotidine, cimetidine, ranitidine hydrochloride, and the like); and

antinauseants/antiemetics (e.g., meclizine hydrochloride, nabilone, prochlorperazine, dimenhydrinate, promethazine hydrochloride, thiethylperazine, scopolamine, and the like).

This list is not intended to be limiting. Additional agents contemplated for delivery employing the devices and methods described herein include agents useful for the treatment of diabetes (e.g., activin, glucagon, insulin, somatostatin, proinsulin, amylin, and the like), carcinomas (e.g., taxol, interleukin-1, interleukin-2 (especially useful for treatment of renal carcinoma), and the like, as well as leuprolide acetate, LHRH analogs (such as nafarelin acetate), and the like, which are especially useful for the treatment of prostatic carcinoma), endometriosis (e.g., LHRH analogs), uterine contraction (e.g., oxytocin), diuresis (e.g., vasopressin), cystic fibrosis (e.g., Dnase (i.e., deoxyribonuclease), SLPI, and the like), neutropenia (e.g., GCSF), MS (e.g., beta 1-interferon), respiratory disorders (e.g., superoxide dismutase), RDS (e.g., surfactants, optionally including apoproteins), obesity (e.g., leptins) and the like.

It will be readily apparent to those skilled in the relevant arts that other suitable modifications and adaptations to the methods and applications described herein may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

### Example 1

### Preparation of Hydrogels

Swellable hydrogels may be prepared according to the methods disclosed in Martin et al., Biomaterials 19: 69-76 (1998). For example, a poly-co-(6-acryloyl-β-methylgalactopyranoside/2,6-diacryloyl-β-methylgalactopyranoside) hydrogel is synthesized by dissolving 2.0 g 6-acryloyl-β-methylgalactopyranoside in 11.3 mL water. 10.9 mg 2,6-diacryloyl-β-methylgalactopyranoside (0.5 mol %) is added. 10 mg 2-2′-azobis[2-(2-imidazolin-2-yl)propane] dihydrochloride (AIPD) is added, and dissolved oxygen is removed by aspiration. The solution is placed in a 55° C. water bath, with nitrogen passing through the system at a rate of 3 mL/min. Polymerization is allowed to proceed for 25 minutes. Size of particles may be regulated varying the stirring rate of the solution. Residual unreacted materials are removed by washing the resulting hydrogel. Other hydrogels may be prepared by using varying amounts of 2,6-diacryloyl-β-methylgalactopyranoside (from 0.05 to 10 mol %), resulting in different swelling properties and, therefore, different pore sizes in the hydrogel. Hydrogels are dried in vacuo at 60° C., and ground if necessary to obtain particles of a particular size.

### Example 2

### Incorporation of Molecules within a Hydrogel

Hydrogels are placed in phosphate buffer for hydration. A balance between the osmotic pressure within the hydrogel and the polymer elasticity sets the physical dimension of the hydrogel particle. Neutralizing the gel tends to reduce the osmotic pressure, and hence the swelling. In this manner, the relative pore size of the hydrogel may be controlled to an extent. The molecule to be incorporated is added to the solution and permitted to enter the particle. After several hours, the particles are dried by lyophilization. If desired, the dried particles may be coated with a mucoadhesive, such as carboxymethylcellulose, carbopol, polycarbophil, tragacanth and sodium alginate. This coating may further serve as an attachment layer for associating a targeting moiety with a particle.

### Example 3

### Preparation of Nanoparticles

Nanoparticles may be prepared according to the methods disclosed in Konan et al., Intl. J. Pharmaceutics 233: 239-52 (2002). For example, 5 g of poly(D,L-lactide-co-glycolide (PLGA) in tetrahydrofuran is added with stirring to 20 g of an aqueous phase of polyvinyl alcohol and 60% w/w magnesium chloride. 60 mL water is added. Particle size is dependent on the stirring rate and time, and the molecular weight of the polyvinyl alcohol. Particle size is measured using a Coulter NanoSizer™ spectroscopy unit. Molecules to be incorporated into the particles may be added prior to polymerization.

Alternatively, nanoparticles may be made according to the methods disclosed in U.S. Pat. No. 5,962,566. For example, 225 mg of a poly (D,L)lactide-co-glycolide copolymer (50 mol %; Mn: 20,000; designated as PLGA) and 225 mg of a polyoxyethylene-polyoxypropylene-polyoxyethylene triblock copolymer (Poloxamer: Pluronic F68) is dissolved in 5 ml of methylene chloride at room temperature for 15 min. The organic solvent is evaporated under nitrogen. Cholesterol 3-sulfate (25 mg) is added to the polymer blend followed by 50 ml of DMSO (p.a.). Dissolution took place for 25 min. at room temperature. A polypeptide of interest is added to the organic solution and dissolved (5 min). This organic solution is dispersed for 2 min. in 700 ml of water with a rotor-stator equipment (Ultra-turrax; IKA Werke, Janke and Kunkel GmbH & Co. KG, Staufen, Germany, S25N-18g drive unit, 24,000 rpm). The suspension obtained is diluted by water until a total volume of 2 l, and then purified by ultrafiltration by using a tangential ultrafiltration equipment (Spiral membrane Amicon, Inc., Beverly, Mass., USA, model S1Y100) and a peristaltic pump (model CH2, Amicon, inlet flow rate: 2 l/min; inlet and back-pressure : respectively: 10 and 8 psi). After a first diafiltration cycle, nanoparticles are diluted in water (2 l) and purification was repeated in order to completely eliminate DMSO and the free protein.

### Example 4

### Preparation of Liposomes

Liposomes may be prepared according to the methods disclosed in U.S. Pat. No. 6,210,707. For example, a suspension of lipid-DNA microparticles (measuring 410.+−.150 nm in size by dynamic laser scattering) composed of plasmid DNA (pCMV/IVS-Luc.sup.+; 10 .mu.g/mL), dimethyl dioctadecylammonium bromide (DDAB, 60 nmol/mL), and dioleoyl phosphatidylethanolamine (DOPE, 60 mnol/mL) in 5% aqueous dextrose, was prepared as described by Hong et al. (FEBS lett. 400:233-237, 1997). An antibody conjugate was prepared by co-incubation of Mal-PEG-DSPE and anti-HER2 antibody Fab′ fragments at a molar ratio of 4:1, at a concentration of the protein of 0.3 mg/mL in aqueous physiological buffer, at pH 7.2 for 2 hours. Lipid-DNA microparticles with conjugated anti-HER2 Fab′ fragments were prepared by incubation of. the lipid-DNA microparticles with the conjugate in the amount of 0.5 mol. % relative to total particle lipid content for at least 30 min. at room temperature. Control particles with linker alone (non-targeted control) were prepared in the similar manner, but non-conjugated, β-mercaptoethanol-quenched Mal-PEG-DSPE was substituted for the Fab′-PEG-DSPE conjugate.

### Example 5

### pIgR Binding Assays

The ability of a designed nanoparticle of the invention to bind different components of pIgR and to undergo endocytosis, transcytosis, and/or exocytosis is preferably examined using the following methods. The ability of a given pIgR-targeting element to bind to pIgR, whether as a separate entity, or as part of a designed protein or designed nanoparticle, is assessed.

Ex Vivo Testing of Ligand Binding

The ex vivo pIgR binding capacity of a designed protein or a designed pIgR-targeted nanoparticle is assessed by measuring endocytosis or transcytosis of bound ligand in mammalian epithelial cells. Receptor-mediated endocytosis provides an efficient means of causing a cell to ingest material which binds to a cell surface receptor. (See Wu et al., J. Biol. Chem. 262:4429-4432, 1987; Wagner et al., Proc. Natl. Acad. Sci. USA 87:3410-3414, 1990, and published EPO patent application EP-A1 0388758). Any number of well known methods for assaying endocytosis may be used to assess binding. For example, binding, transcytosis, and internalization assays are described at length in Breitfeld et al. (J. Cell Biol. 109:475-486, 1989).

Ligand-pIgR binding is measured by a variety of techniques known in the art, e.g., immunoassays and immunoprecipitation. By way of example, antibodies to the biologically active portion of a designed nanoparticle are used to bind and precipitate detectably labeled pIgR molecules; the amount of labeled material that is precipitated corresponds to the degree of pIgR binding to a ligand such as, e.g., a protein conjugate having a pIgR-targeting element (see Tajima, J. Oral Sci. 42:27-31, 2000). GST-domain 6 may be absorbed to a plastic well and used to capture designed nanoparticte that contain a pIgR-binding functional unit. After the plate has been washed and prepared, the designed nanoparticle is incubated with the GST-domain 6 to allow capture of the designed nanoparticle. If the designed nanoparticle contains a functional unit(s) that binds to domain 6, then it will be specifically bound to the well. The excess reagents are washed free in the well, and a second detection component or components is added. For example, an antibody that recognizes the pIgR binding element is used. Or an antibody that recognizes another element of the designed protein which is part of the designed nanoparticle can be used. Or an antibody that recognizes the nanoparticle itself can be used. In any case, the antibody is linked covalently to an enzyme, fluorescent molecule, or other entity that is capable of being detected. For example, an antibody linked to horse radish peroxidase can be used. After incubation with the antibody detecting system, a substrate of horse radish peroxidase, such as TMB, is added and the color allowed to develop under tightly controlled conditions. The amount of TMB that was modified by the horse radish peroxidase is quantitated by reading its absorbance and the amount is directly proprotional to the amount of pIgR binding designed protein adsorbed to the well.

Other formats are possible and are known to those skilled in the art. For example, beads instead of plastic wells may be used to immobilize the capture entity (in this example, GST-domain 6). Additionally, the nanoparticles may contain fluorescently labelled compounds or radioactive compounds that are easily detected.

Apical Endocytosis is measured by binding a ligand such as a Fab fragment to the stalk at the apical surface of pIgR-transfected Madin-Darby canine kidney (MDCK) cells at 4° C., warming to 37° C. for brief periods (0-10 min), and cooling the cells to 4° C. In addition to Fab fragments, other binding entities are used, including sFv, F(ab′)2, diabodies, scABs, peptides selected by phage display, etc., all of which may be part of a designed nanoparticle as described herein. Methods of pIgR expression in MDCK cells are known in the art (Breitfeld et al., Methods in Cell Biology 32:329-337, (1989). Designed proteins and designed nanoparticles that react with pIgR or the stalk of pIgR and that remain on the surface are removed by stripping at pH 2.3. Intracellular designed proteins and designed nanoparticles are those that remain cell-associated after the stripping, while surface-bound designed proteins and designed nanoparticles are those removed by the acid wash. Controls for non-specific sticking include using control designed proteins that have been purposely modified or mutated so as not to bind to pIgR or pIgR stalk and/or MDCK cells that are not transfected with pIgR or derivatives of pIgR including the stalk of pIgR.

Apical to Basolateral (“Reverse”) Transcytosis is assessed using MDCK cells or MDCK cells that have been transfected with pIgR derived from various animal species. pIgR that is transfected may contain the entire pIgR molecule with its transmembrane portion or only portions of the molecule. Various parts, portions, or domains of pIgR may be assembled into chimeras so as to result in a pIgR molecule that is partly one animal and partly another animal. pIgR may be assembled to contain amino acid sequences that are derived from two or more animal species. The protein or the nanoparticle that binds to pIgR is reacted with the control cells or the cells containing pIgR at the apical surface at 4° C., the cells are warmed up to 37° for 0 to 240 min, and then measured for the amount of reactive designed protein or designed nanoparticle delivered into the basolateral medium. This basolaterally-delivered protein or nanoparticle is compared to the sum of protein or nanoparticle that remains associated with the cells (intracellular or acid-stripped) and the designed protein or designed nanoparticle released back into the apical medium. Alternatively, transcystosis is assessed by continuously exposing cells to the protein or nanoparticle in the apical medium and measuring accumulation of protein or nanoparticle in the basolateral medium. This method avoids cooling the cells, but does not provide the kinetics of transporting a single cohort of ligand. In both methods, degradation of the protein or nanoparticle can be assessed by running aliquots of the transcytosed designed protein on SDS-PAGE and probing a Western blot with appropriate antibodies.

Basolateral Endocytosis is assessed by methods such as those described by Tajima (J. Oral Sci. 42:27-31, 2000). Non-specific transport (e.g. due to fluid phase endocytosis and transcytosis, or paracelfular leakage between cells) can be controlled for by using MDCK cells that are not transfected with the pIgR.

In Yivo Testing of Ligand Binding

Transcytosis is assessed using pathogen-free experimental animals such as Sprague-Dawley rats. Detectably labeled ligand (e.g. a radioiodinated antibody) is administered into, e.g., the nares (the pair of openings of the nose or nasal cavity of a vertebrate) or the intestine (see Example 14). As will be understood by those of skill in the art, a “detectable label” is a composition or moiety that is detectable by spectroscopic, photochemical, biochemical, immunochemical, electromagnetic, radiochemical, or chemical means such as fluorescence, chemifluoresence, or chemiluminescence, or any other appropriate means

In vivo Apical to Basolateral (“Reverse”) Transcytosis is assessed by measuring the delivery of a pIgR-targeting protein into the circulation as measured by the presence of a detectable label that has been incorporated into the protein that is being tested. The integrity of the ligand recovered from the circulation can be assessed by analyzing the ligand on SDS polyacrylamide gel electrophoresis.

Measurement of the delivery of the therapeutic entity by determination of its concentration in blood

A cannula is implanted into the jugular vein of an animal (e.g., a rat) for the purpose of collecting blood samples at various times. Another cannula is implanted into a region of the intestine, jejunum, ileum, or colon, for the purpose of administering the therapeutic entity to the intestine. A 350-375 gram Spraque-Dawley rat is suitable for this purpose. Other strains of rats may also be used. The cannula are guided under the skin so that they exit the skin directly between the shoulders of the rat. This position does not allow the rat to damage the cannula. A single rat per cage is required. The rat is administered the therapeutic entity 2 to 7 days after the cannulae are implanted. During this time, the rat is observed for its general health and to determine the patency of the cannulae.

The therapeutic entity is given to the rat through the intestinal cannula. Before administration, a sample of blood (approximately 200 microliters) is withdrawn through the jugular vein cannula. Samples of blood arc collected over a 8 to 72 hour period. The jugular cannula is kept patent by using saline with a small amount of heparin to prevent clotting. The blood is collected into a 1.5 ml Eppendorf tube that contains 5 microliters of heparin to prevent clotting. The blood is kept on ice for up to 1 hour, but no longer, before it is centrifuged in a table top Eppendorf centrifuge for 30 to 60 seconds. The supernatant is collected (plasma) and stored in a suitable manner, usually by freezing at −80 degrees C.

Pulmonary delivery may be effected by instillation of the designed nanoparticle through a thin tube (or “cannula”) inserted into the trachea of an anesthetized subject, such as a rat, mouse, rabbit, or primate. A bronchoscope may be used to instill the designed nanoparticle into the lung of an anesthetized subject. Blood samples may be obtained and analyzed as described above. Methods to aerosolize the material are known to those skilled in the art. Nebulizers are a preferred method of administration. Various commercial nebulizers (e.g. Pari Respiratory Equipment, Inc.) are available.

The presence and amount of the therapeutic entity is measured using any convenient assay. For example, the therapeutic entity may be radioiodinated using ¹²⁵I, introduced using any of the usual methods of radioiodination that are known to those skilled in the art. These methods include using chloramine T, ICI, galactoperoxidase beads, or lodo-beads. The therapeutic entity that has been radioiodinated is separated from unreacted ¹²⁵I by chromatography, including size separation on Sephadex or Sepharose, or by dialysis. The weight of the blood is determined by collecting the blood into a preweighed Eppendorf or small glass tube and determining the weight of the blood by subtraction after weighing the tube containing the blood. The entire tube is counted in a gamma counter and the number of counts per minute divided by the weight of the blood to determine the number of cpm per gram of blood (essentially equivalent to the cpm/ml of blood). A graph of the cpm/ml of blood as a function of time after administration of the radiolabelled therapeutic entity corresponds to the transport of the therapeutic entity from the intestine or pulmonary tract (e.g., lung) into blood.

The therapeutic entity may also be examined to determine if it has the correct molecular weight by SDS-PAGE. A sample of the plasma is compared on SDS-PAGE with a sample of the radiolabelled therapeutic entity that was administered through the cannula. If the patterns of radioactivity (autoradiography) are the same, then it may be concluded that the therapeutic entity that is present in blood is not degraded. The blood sample may be reacted to immunoprecipitate the therapeutic entity. The immunoprecipitated sample may be compared to a sample of immunoprecipitated from the stock radiolabelled therapeutic entity by displaying them on SDS-PAGE. A quantitative estimate of the amount of therapeutic entity may be made by comparing the amount of cpm that was immunoprecipitated from blood samples and from stock radiolabelled therapeutic entity.

An ELISA may also be used to determine the concentration of the therapeutic entity. In this case, the therapeutic entity has not been radiolabelled. An antibody that recognizes the therapeutic entity is coated to the bottom of 96-well plates. After washing, the presence and quantity of the therapeutic entity is determined by reacting it with a second antibody that is conjugated to horse radish peroxidase or alkaline phosphatase. After washing, a substrate for horse radish peroxidase or alkaline phosphatase is incubated in the well and the amount of the product determined by spectrophotometry at an appropriate wavelength. A control or standard curve (using known quantities of the therapeutic entity) is used to determine the concentration of the therapeutic entity in the plasma samples. Depending on the drug or other agent loaded into the designed nanoparticle, various HPLC methods may also be used to detect and to quantitate the drug in plasma.

Determining the Effectiveness of the Therapeutic Entity

The effectiveness of designed nanoparticle conjugates can be tested in appropriate animal models. Rat cancer models may be used for determining the efficacy of designed proteins or designed nanoparticles. For example, a designed nanoparticle that is loaded with drug(s) effective against cell growth, or the equivalent nanoparticle that does not carry such drugs, are tested for the ability to inhibit the growth of tumors implanted into a rat. In the case in which an element incorporated into the nanoparticle that reacts with rat tumor cells and inhibits or prevents their growth, the designed nanoparticle loaded with that element inhibit the growth of the tumor cells implanted into the rat in a detectable way. The cells that are implanted may be of rat origin and grown in a wild type rat. In the case in which the element incorporated into the nanoparticle reacts with human tumor cells, the tumors cells that are implanted may be of human origin and grown in an immune compromised rat (scid). The differences in growth characteristics of the different treatments indicate the effectiveness of the delivery of the materials.

Alternatively, a designed nanoparticle comprising erythropoetin (“EPO”) may be tested for the ability to stimulate erythropoiesis via stimulating the division and differentiation of committed erythroid progenitor cells. Therapeutic designed nanoparticles may be administered by instillation or inhalation into the pulmonary tract (e.g., the bronchus, the trachea, the lung, etc.). Stimulation of erythropoiesis by a therapeutically effective dose of EPO may be monitored by observing patient samples for an increase in reticulocyte counts, a rise in hematocrit, or a rise in hemoglobin levels.

The animal (e.g., rat) is prepared for administration of the therapeutic entity by inserting a cannula into a region of the intestine, such as the jejunum, ileum, or colon. After the surgery required to insert the cannula, the animal is optionally rested for 2 to 7 days to recover. During this time, the animal may be observed for the patency of the cannula and for its general health. During this time, or shortly before the surgery, tumor cells are injected subcutaneously into the flank of the animal. Depending on the specific tumor cell line used and its ability to form tumors, 10,000 to 5,000,000 cells are injected subcutaneously. The cells are first grown in tissue culture medium and taken up as a suspension. The tumor cells are allowed to grow for 5 to 14 days before the tumor is treated with the therapeutic entity, administered e.g. through an intestinal cannula, or by aerosol through the pulmonary or nasal cavity.

Tumorigenic cells for use in the foregoing method include, but are not limited to, the following. MTLn3, a rat mammary adenocarcinoma line that grows after subcutaneous injection (Toyota el al., Intnatl. J. Cancer 76, 499-505, 1998); EGFR-expressing cell line TE8, an esophageal squamous cell carcinoma, and EGFR-deficient cell line H69 (Suwa, T., Ozawa, S., Ueda, M., Ando, N., and Ktiajima, M., International Journal of Cancer 75:626-34, 1998); A431 cells, a human epidermoid carcinoma tumor cell line grown in athymic rodents, including rats; LNCAP tumors implanted subcutaneously in athymic nude rats (Rubenstein, M., Mirochnik, Y., Ray, V., and Guinan, P., Medical Oncology 14:131, 1997); and C6 cells implanted stereotactically into the right caudate nucleus of Wistar rats.

Measurements of the tumor size are made using calipers to measure the dimensions of the tumor in two directions. The volume of the tumor may be determined by multiplying the longest dimension times the square of the shortest dimension and dividing the product by 2. By plotting the tumor volume as a function of time (using the average or mean tumor volume) for a group of rats given the therapeutic entity and comparing the same plot for a group of untreated rats bearing the same tumor, one skilled in the art may determined the ability of the therapeutic entity to inhibit the growth of the tumor or even to eradicate the tumor. The measurements may be interpretable for a period as short as a few days after administration of the therapeutic entity.

Tumor cells, such C6 cells, are implanted stereotactically into the right caudate nucleus of Wistar rats. A cannula into the intestine may also be put into these rats for the purpose of administering the therapeutic entity. Rats with well-established cerebral C6 glioma foci may be given the therapeutic entity through the intestinal cannula The mean survival time of tumor bearing rats is about 15-20 days in this model. The efficacy of the therapeutic entity may be measured by comparing the live span of control rats (tumor bearing rats given no therapeutic entity) to the rats given the therapeutic entity. [Inhibitory effect of antisense epidermal growth factor receptor RNA on the proliferation of rat C6 glioma cells in vitro and in vivo. Journal of Neurosurgery. 92:132-9, 2000].

The inventions illustratively described herein may suitably be practiced in the absence of any element or elements, limitation or limitations, not specifically disclosed herein. Thus, for example, the terms “comprising”, “including,” “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

The contents of the articles, patents, and patent applications, and all other documents and electronically available information mentioned or cited herein, are hereby incorporated by reference in their entirety to the same extent as if each individual publication was specifically and individually indicated to be incorporated by reference. Applicants reserve the right to physically incorporate into this application any and all materials and information from any such articles, patents, patent applications, or other documents.

1. A method of preparing a therapeutic composition comprising a targeting moiety that specifically binds to a cell surface component that promotes active transport, endocytosis or transcytosis, and a therapeutic moiety, comprising: physically entrapping (i) a portion of said targeting moiety, or an anchor moiety that binds to said targeting moiety, and (ii) said therapeutic moiety, within a particle having physical dimensions compatible with cellular uptake, whereby said particle is adapted to specifically bind to said cell surface component.

2. A method according to claim 1, wherein said cell surface component is present on epithelial cells.

3. A method according to claim 2, wherein said epithelial cells are enterocytes.

4. A method according to claim 1, wherein said cell surface component is present on endothelial cells.

5. A method according to claim 1, wherein said targeting moiety and said therapeutic moiety are not bound to one another.

6. A method according to claim 1, wherein said targeting moiety and said therapeutic moiety are covalently or noncovalently bound to one another.

7. A method according to claim 1, wherein said targeting moiety is selected from the group consisting of a polypeptide, a recombinant polypeptide, an antibody, an antibody fragment, a single-chain variable region fragment, a small molecule, an oligonucleotide, an oligosaccharide, a polysaccharide, a cyclic polypeptide, a peptidomimetic, and an aptamer.

8. A method according to claim 1, wherein said cell surface component is selected from the group consisting of pIgR, transferrin receptor, vitamin B12 receptor, FcRn, an integrin, Flt-1, Flk-1, Flt-4, and low density lipoprotein receptor.

9. A method according to claim 1, wherein said therapeutic moiety is selected from the group consisting of a polypeptide, a recombinant polypeptide, an antibody, an antibody fragment, a single-chain variable region fragment, a small molecule, an oligonucleotide, an oligosaccharide, a polysaccharide, a cyclic polypeptide, a peptidomimetic, and an aptamer.

10. A method according to claim 1, wherein upon said physical entrapment, said anchor moiety comprises a first region entrapped within said particle and a second region protruding from the surface of said particle for binding to said targeting moiety.

11. A method according to claim 10, wherein said first region is selected from the group consisting of a polypeptide, a recombinant polypeptide, a nucleic acid, a poly (ethylene oxide), a peptidomimetic, a cyclic peptide, a oligosaccharide, a polysaccharide, and a dextran.

12. A method according to claim 10, wherein said second region is selected from the group consisting of a polypeptide, an antibody, an antibody fragment, a single-chain variable region fragment, a small molecule, an oligonucleotide, an oligosaccharide, a polysaccharide, a cyclic polypeptide, a peptidomimetic, and an aptamer.

13. A method according to claim 12, wherein said second region is a polypeptide sequence that forms a coiled-coil with a complementary polypeptide sequence on said targeting moiety.

14. A method according to claim 1, wherein upon said physical entrapment, said targeting moiety comprises a first region entrapped within said particle and a second region protruding from the surface of said particle that specifically binds to said cell surface component.

15. A method according to claim 14, wherein during said physical entrapment step, said particle comprises pores having physical dimensions capable of accepting said first region, but incapable of accepting said second region.

16. A method according to claim 15, wherein said pores are produced by swelling said particle, and wherein said targeting moiety is entrapped by reducing said swelling.

17. A method according to claim 1, wherein said therapeutic moiety is entrapped within said particle by polymerization of material forming said particle.

18. A method according to claim 1, wherein said portion of said targeting moiety, or said anchor moiety, is entrapped within said particle by polymerization of material forming said particle.

19. A method according to claim 1, wherein said portion of said targeting moiety, or said anchor moiety, and said therapeutic moiety are entrapped within said particle by polymerization of material forming said particle.

20. A method according to claim 6, wherein upon said physical entrapment, said therapeutic moiety is entrapped within said particle and all or a portion of said targeting moiety is protruding from the surface of said particle.

21-60. (canceled)
